(19) United States
(12) Patent Application Publication
(10) Pub. No.: US 2011/0117209 A1
(43) Pub. Date: May 19, 2011

(54) COMPOSITION FOR DISINFECTION AND DECONTAMINATION OF BODIES CONTAMINATED BY PRIONS AND CONVENTIONAL TRANSMISSIBLE AGENTS

(75) Inventors: Bertrand Letartre, Lille Hellemmes (FR); Sébastien Terrier, Lille Hellemmes (FR); Gaëtan Rauwel, Lillc Hcllcmmcs (FR); Jacques Criquelion, Lille Hellemmes (FR)

(73) Assignee: WALMEC Spa, Milano (IT)

(21) Appl. No.: 12/996,202

(22) PCT Filed: Jun. 5, 2009
(86) PCT No.: PCT/FR2009/000659

§ 371 Date: Dec. 3, 2010

(30) Foreign Application Priority Data
Jun. 5, 2008 (FR) 08/03130

(51) Int. Cl. A01N 59/20; A01P 1/00

(52) U.S. Cl. 424/616

(57) ABSTRACT

The invention relates to a composition for the simultaneous disinfection and decontamination of bodies contaminated by CTAs such as bacteria, yeasts, mould spores, viruses or others, and NCTAs such as prions. According to the invention, the composition includes a hydrogen peroxide solution and an aqueous solution of copper or a derivative thereof such as copper salts, and at least one anionic salified surfactant compound inhibiting the decomposition of hydrogen peroxide in the presence of copper. Said composition can be prepared extemporaneously from a mixture of an aqueous copper solution and of an aqueous hydrogen peroxide solution. The invention further relates to an aqueous copper solution and to an aqueous hydrogen peroxide solution to be mixed together.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

Disclosed is a composition for simultaneous disinfection and decontamination of bodies contaminated by conventional transmissible agents (CTAs) such as in particular bacteriae, yeasts, mould spores, mycobacteriae, viruses, bacteria spores, and non conventional transmissible agents (NCTAs) such as prions.

The invention will especially find a particular application for the treatment of heat sensitive objects such as endoscopes.

2. Description of Related Art Including Information Disclosed Under 37 CFR 1.97 and 37 CFR 1.98.

When a multi-usage object is suspectedly infected by a conventional or non-conventional transmissible agent, it is recommended as follows:
it should be destroyed by incineration if economic data so enable, and in the reverse case,
it should be sterilised by steam (autoclaving) if heat-resistant, if not,
it should be treated by a so-called disinfection and/or decontamination treatment.

The invention described here focuses more particularly on the third situation when the object is especially heat sensitive and multi-usage.

An object which might be contaminated by a CTA and/or a NCTA results from a use of this object in internal and/or external contact with a body. The case of using an endoscopic material during an exploratory medical examination may be mentioned. In contact with certain organs and/or human or animal organic fluids, the object can be contaminated by NCTAs but also, at the same time, by CTAs.

It is recommended between examinations on two different patients to perform operations so as to control the NCTA and CTAs-induced infectious risks.

Until now, there is no simple and efficient procedure for jointly disinfecting against CTAs and decontaminating against NCTAs, multi-usage heat sensitive objects with sensitive alkaline pH.

The procedure for disinfecting multi-usage heat sensitive objects by immersion is known and practiced currently. The procedure for decontaminating NCTA objects, by immersion, by alkaline to very alkaline pH procedures (a solution of sodium hydroxide or of potassium hydroxide or of polyethanolamine with pH ranging from 10 to 14) is also known. Until now, no procedure by immersion for decontaminating alkaline pH-sensitive objects against NCTAs has been implemented.

Document WO 2005/118762 however discloses the use of copper and its derivatives, for the decontamination of products contaminated by prions. The decontamination treatment is realised preferably with a solution containing said compound at a rate of at least 500 micromoles of metal derivatives This solution may contain hydrogen peroxide (H₂O₂) in the solution at a rate of approximately 50 millimoles.

Such a composition thus enables to decontaminate bodies soiled by prions but does not permit, by reason of the low proportion in H₂O₂, to disinfect CTAs such as in particular bacteriae, yeasts, viruses or similar, with an efficiency validated according to the European standards (e.g. EN 14561, EN 14562, EN 14563, EN 14476 . . . ).

It is particularly difficult to identify a priori the risk of NCTA-induced contamination. Only apparent pathological signs or a history documenting the medical profile of an individual may enable to fathom the risk. Systematic decontamination of any object which might be contaminated by NCTAs is desirable for limiting any risk of cross contamination.

For easier management of a decontamination operation and to make feasible using the procedures for cleaning and disinfecting the objects, the aim of the present invention is to conduct decontamination against NCTAs along with current cleaning and disinfection operations.

More particularly, the aim of the present invention is to provide a composition for simultaneous disinfection and decontamination of bodies contaminated by conventional transmissible agents (CTAs) and non conventional transmissible agents (NCTAs) such as prions.

Another aim of the invention is to provide such a composition which is particularly suitable for manual treatment, especially by dipping.

Another aim of the invention is to provide a method of preparing such a composition from two solutions, respectively based on copper and based on hydrogen peroxide.

Another aim of the invention is to provide an aqueous copper solution for quick preparation of a composition according to the invention, in mixture with an aqueous hydrogen peroxide solution.

Another aim of the present invention is to offer an aqueous hydrogen peroxide solution to be mixed with an aqueous copper solution for the realisation of a composition according to the invention.

Other aims and advantages of the present invention will appear in the following description which is given only by way of example and without being limited thereto.

### BRIEF SUMMARY OF THE INVENTION

Disclosed is first of all a composition for simultaneous disinfection and decontamination of bodies contaminated by conventional transmissible agents (CTAs) such as in particular bacteriae, yeasts, mould spores, mycobacteriae, bacteria spores, viruses and non conventional transmissible agents (NCTAs) such as prions, particularly for the treatment of heat sensitive objects such as endoscopes.

According to the invention, said composition includes a solution of hydrogen peroxide and an aqueous solution of copper or of one of the derivatives thereof such as copper, as well as at least one anionic salified surfactant compound inhibiting the decomposition of hydrogen peroxide in the presence of copper.

The invention also relates to an aqueous copper solution to be mixed with an aqueous hydrogen peroxide solution for the realisation of the composition according to the invention.

The aqueous copper solution according to the invention comprises at least one anionic salified surfactant compound inhibiting the decomposition of hydrogen peroxide in the presence of copper.

The invention also relates to an aqueous hydrogen peroxide solution to be mixed with an aqueous copper solution for the realisation of the composition according to the invention.

The aqueous hydrogen peroxide solution according to the invention contains hydrogen peroxide-stabilising agents selected among phosphonates, perborates with a content ranging between 0.002% and 0.07% in weight.

There is also described a method of preparing the composition according to the invention wherein an aqueous copper solution is mixed extemporaneously with an aqueous hydrogen peroxide solution.

There is further disclosed the application of the composition according to the invention and/or of the aqueous copper solution in mixture with a hydrogen peroxide solution for simultaneous disinfection and decontamination of bodies contaminated by CTAs such as in particular bacteriae, yeasts or similar and NCTAs such as prions.

The invention will be understood better when reading the following description.

### DETAILED DESCRIPTION OF THE INVENTION

The activity of certain metals especially copper, used in the form of salt, has been demonstrated for efficiently inactivating pathogenous agents such as non conventional transmissible agents. The efficiency as regards these agents can be improved in the presence of peroxide, in particular hydrogen peroxide. Unfortunately, both these compounds have a very attracting character towards one another: copper salt, such as copper sulfate, is a reducing compound, whereas hydrogen peroxide is a powerful oxidiser.

Peroxides are active on CTAs in high concentration to satisfy the assessment standard of the biocidal activities for acceptable times in medical or industrial practice. The concentrations of hydrogen peroxide may range from 1 to 10% according to the requested action time. In the case of this method and to be in adequation with the practices in the domain of health or industry which call for short treatment times, such as 10 to 30 minutes, the most relevant concentration of hydrogen peroxide may be approximately 6.5 to 8.5%. These high concentrations of oxidiser especially strengthen the incompatibilities between oxidiser and reducer of the hydrogen peroxide/copper salt association.

The invention hence comprises more particularly in making these reducing and oxidising compounds both compatible and useable, which are intrinsically incompatible, so as to maintain the coupled disinfection and decontamination efficiencies against NCTAs and CTAs.

The simple mixture of copper salt and of hydrogen peroxide causes quasi instant decomposition of the peroxides with release of oxygen. Both these compounds should be isolated in two distinct vessels in order to propose a useable method with at least a minimal stability and hence sales duration. This sole necessary approach is however not sufficient since the objective is to secure stability, hence a usage of the solution after mixture of the vessels containing the aqueous copper solution and the hydrogen peroxide solution respectively.

Reconstitution by simple mixture will cause said decomposition, instantly initiated and leading to partial to total degradation of hydrogen peroxide: the consequence of this mixture is partial to total loss of efficiency as regards NCTAs and CTAs as described in table 1:

TABLE 1
stability of hydrogen peroxide in the presence of copper sulfate

| | T0 | T = 15 min | T = 24 hours | T = 72 hours |
|---|---|---|---|---|
| H2O2 | 7.5% | 7.4% | 2.62% | 0.37% |
| concentration | | | | |
| (weight) | | | | |

TABLE 1bis
compared activities of hydrogen peroxide, of copper
salts and of the mixture according to the invention along
the standard EN 13727 as regards Staphylococcus aureus.

| | | CuSO4 | Mixture of the invention | | | |
|---|---|---|---|---|---|---|
| | H2O2 | (0.5 | T = 30 | | | |
| | 7.5% | millimoles) | min | T = 24 h | T = 72 h | T = 7 days |
| Staph- | Active | Inefficient | Active | Active | Ineffi- | Ineffi- |
| ylococcus | >5 log | | >5 log | >5 log | cient | cient |
| aureus | | | | | | |

The use of components salified by soda or potash among which sodium alkylbenzenesulfonate, sodium alkylethersulfate, sodium alkylsulfate, interferes with the decomposition reaction of hydrogen peroxide by copper salts. As demonstrated in table 2, the quantity and the identity are primordial to manage the interaction between both components. Alkylsulfate and sodium alkylbenzenesulfonate are especially two salts having good performances. The C12 alkyl chain has been assessed particularly.

TABLE 2
stability of hydrogen peroxide in the presence of copper salts dependent
on the concentration (% weight) of anionic component.

| | Anionic | | T = | T = | |
|---|---|---|---|---|---|
| Data +/−0.1% | concentrations | T0 | 15 min | 24 h | T = 72 h |
| Sodium | 0.1% | 7.6% | 7.6% | 7.3% | 6.9% |
| alkylethersulfate | 1% | 7.6% | 7.6% | 7.6% | 7.6% |
| Sodium alkyl | 0.1% | 7.6% | 7.6% | 7.4% | 7.3% |
| benzene | 1% | 7.5% | 7.5% | 7.5% | 7.5% |
| sulfonate | | | | | |
| Sodium alkyl | 0.1% | 7.6% | 7.6% | 7.6% | 7.4% |
| (dodecyl) | 1% | 7.6% | 7.6% | 7.6% | 7.6% |
| sulfate | | | | | |

The invention also relates to a composition for simultaneous disinfection and decontamination of bodies contaminated by conventional transmissible agents (CTAs) such as in particular bacteriae, yeasts, mould spores, mycobacteriae, viruses, bacteria spores, and non conventional transmissible agents (NCTAs) such as prions, particularly for the treatment of heat sensitive objects such as endoscopes.

By bodies is meant here any object, in particular multi-usage and heat sensitive objects, or still any surface such as in particular floor or work surface.

According to the invention, the composition includes a solution of hydrogen peroxide and an aqueous solution of copper or of one of the derivatives thereof such as copper, as well as at least one anionic salified surfactant compound inhibiting the decomposition of hydrogen peroxide in the presence of copper.

According to an embodiment, the composition may exhibit (% in weight):
anhydrous copper salts between 0.005% and 0.01%,
hydrogen peroxide between 5% and 9%,
anionic salified surfactant compound between 0.5% and 1.5%,
demineralised water q.s. 100%, the sum of the components being equal to 100%.

For assigning anti-microbial properties without altering the prionicidal properties of the composition, the concentration of hydrogen peroxide may range between 5% and 9% in weight.

Said at least one anionic salified surfactant compound may be selected among the (non limiting) following group: dodecylbenzenesulfonate, dodecylsulfate, sodium dodecylethersulfate.

The purity of anionic salt is important as illustrated in table 3 for the stability of the oxidising agent.

TABLE 3
stability of hydrogen peroxide in the presence of copper salts dependent
on the purity of anionic salt

| | T0 | T = 15 min | T = 24 h |
|---|---|---|---|
| Industrial quality | 7.5% (+/−0.1) | 7.3% (+/−0.1) | 7.2% (+/−0.1) |
| High level of purity | 7.5% (+/−0.1) | 7.5% (+/−0.1) | 7.5% (+/−0.1) |
| (synthesis) | | | |

Said at least one anionic salified compound must thus be advantageously ultra-pure, of denominated synthesis quality.

The instability of copper salts may show forth in deposits at the air/solution junction of the mixture of the copper solution and of the hydrogen peroxide solution. If said deposits are initiated, the presence of salts, in particular calcium and magnesium salts, in the rinsing water (network water) of the objects after cleaning and before soaking in the composition increases the quantity thereof which may prove penalising during the treatment of objects with small diameter channels.

The presence of copper salts may involve optimisation of their solubility, in particular with oxidisers. Insolubility or partial solubility of copper salts is strengthened by calcium and magnesium salts, among others conveyed by the rinsing water of the objects after cleaning and before soaking in the composition. The components such as alkyl poly glucoside in quantity and in quality may be particularly efficient especially when associated in proportion selected according to the number of carbon of the alkyl chains. According to an embodiment, the composition may also include surfactants such as carefully selected poly glucoside fatty alcohols, such as in particular C4 alkyls, for solubilising copper salts.

The use in the copper solution of large amounts of anionic salts, of alkylpolyglucosides for solubilising copper salts in the presence in particular of calcium salts, magnesium salts or similar, induces an instability and phase-shift phenomenon by over-concentration. The compounds, especially hydrotopic agents, such as sodium sulfonate toluene or sulfonate xylene are known as preferential compounds for remedying these situations.

TABLE 4
presence of deposit according to the presence of compounds such as
alkylpolyglucosides

| | T0 | T = 24 h | T = 7 days |
|---|---|---|---|
| H2O2 solution + | No | Yes | Yes |
| Cu salts + | | | |
| anionic derivatives | | | |
| H2O2 solution + | No | No | No |
| Cu salts + | | | |
| anionic derivatives + | | | |
| alkylpolyglucosides | | | |

The adjunction of compounds such as fatty alcohol alkolyxate selected according to their hydrophilic lipophilic balance, in combination with solubilising agents such as alcohol with a number of carbons in particular ranging between 4 and 6 favourable to this complex mixture, may assign the best balance for stability over time, especially at low temperatures: over 1 year for temperatures ranging from +5° C. to +35° C. The composition may include poly alkoxylated fatty alcohol surfactants for assigning a hydrotopic character in combination with a solubilising agent such as alcohol or polyol, promoting in particular stability to the cold.

The use of a colouring agent can be necessary to differentiate the composition from another solution coexisting on the same table top. The choice of the colouring agent can be specifically studied since it should coexist for a certain time, especially several days in the composition, in particular after mixture of a copper-based solution and of a hydroxide peroxide solution whose oxidising agents are besides known for their discoloration quality. A blue triarylmethane colouring agent may prove a good compromise: it colours the solution with a satisfactory stability over time, without colouring the objects to be treated.

According to an embodiment, the composition may include (% in weight):
copper salts between 0.005% and 0.01%,
hydrogen peroxide between 5% and 9%,
anionic salified surfactant compound between 0.5% and 1.5%,
surfactants such as polyglucoside fatty alcohols between 0.1% and 0.4%,
polyalkoxylated fatty alcohol surfactant 0.1% and 0.3%,
solubilising agent such as alcohols or polyols, between 0.1% and 0.3%,
colouring agent q.s.
demineralised water, q.s. 100%, the sum of the components being equal to 100%.

There is also described a method of preparing the composition according to the invention wherein an aqueous copper solution is mixed extemporaneously with an aqueous hydrogen peroxide solution.

Said aqueous copper solution may be added to the solution of peroxide in the respective 1/25 indicative proportions.

The aqueous copper solution to be mixed with an aqueous hydrogen peroxide solution may according to an embodiment further comprise said at least one anionic salified surfactant compound inhibiting the decomposition of hydrogen peroxide in the presence of copper.

The anionic salified surfactant compound may be selected among the following group: dodecylbenzenesulfonate, dodecylsulfate, sodium dodecylethersulfate, with a content ranging between 1 and 50%, in particular between 20 and 30%.

According to an embodiment, the surfactant compound is dodecylsulfate or sodium dodecylethersulfate with a content ranging between 20 and 30% in weight, for obtaining in particular a final concentration in the composition according to the invention of approximately 1%.

Said at least one anionic salified compound may be ultra-pure of denominated synthesis quality, in particular poor in impurities and other synthesis residues, capable of catalysing degradation of peroxides among others.

The aqueous copper solution may comprise at least one surfactant such as poly glucoside fatty alcohol for solubilising copper salts, with a content ranging between 2.5 and 10% in weight, for obtaining especially a final concentration in the composition according to the invention of approximately 0.1 to 0.4%

The aqueous copper solution may comprise at least one surfactant such as poly alkoxylated fatty alcohol for stabilising the composition, with a content ranging between 2.5 and 7.5% in weight, for obtaining especially a final concentration in the composition according to the invention of approximately 0.1 to 0.3%.

The aqueous copper solution may include a content in copper salts ranging between 0.1% and 0.25% in weight expressed in sulfate, enabling in particular to obtain a final concentration in the reconstructed solution from 0.005% to 0.01%.

The aqueous copper solution may comprise a solubilising agent such as alcohol or polyols with a content ranging between 2 and 10%, especially between 4 and 7%.

The aqueous copper solution may include a colouring agent, especially a blue colouring agent, such as triarylmethane, compatible with metal salts and hydrogen peroxide.

The aqueous copper solution may include (percentage in weight):
copper salts between 0.1% and 0.25%
anionic salified surfactant compound between 20% and 30%,
surfactants such as polyglucoside fatty alcohols between 2.5% and 10%,
polyalkoxylated fatty alcohol surfactant 2.5% and 7.5%,
solubilising agent such as alcohols or polyols between 4% and 7%,
colouring agent q.s.,
demineralised water q.s. 100%, the sum of the components being equal to 100%.

Said copper solution may be added to the solution of peroxides in the respective 1/25 indicative proportions.

The invention also relates to an aqueous hydrogen peroxide solution to be mixed with an aqueous copper solution, especially as described previously, for the realisation of a composition according to the invention.

The oxidising solutions are particularly made brittle by the presence of metal salts. It is thus preferable to protect these oxidising solutions on the one hand by high analytical quality choice, and on the other hand, by the adjunction of phosphonates, sodium or potassium persalts.

The addition may be provided in an amount just sufficient for inerting any mineral or metal component which is undesirable for the aqueous hydrogen peroxide solution on its own or in mixture with the copper based aqueous solution.

According to the invention, the hydrogen peroxide solution contains hydrogen peroxide-stabilising agents selected among phosphonates, perborates with a content ranging between 0.002% and 0.07% in weight.

As demonstrated in table 5, the activity over several microorganisms, according to the European standard EN 13727 and EN 13624, is higher for a composition according to the invention relative to a hydrogen peroxide and copper sulfate solution only.

TABLE 5
compared activity 15 minutes after mixing according to the standards
EN 13727 (5-log reduction of the microbial population in 5 minutes)
and EN 13624 (4-log reduction of the microbial population in
15 minutes) at +20° C.

| | Pseudonomas aeruginosa | | Candida albicans | |
|---|---|---|---|---|
| | EN 13727 | | EN 13624 | |
| | % aqueous | | % aqueous | |
| | copper solution | Reduced | copper solution | Reduced |
| | (in volume) | activity | (in volume) | activity |
| Hydrogen | 20% | <4 log | 20% | <2.7 log |
| peroxide | 40% | >5.4 log | 40% | >4 log |
| solution + | | | | |
| copper sulfate | | | | |
| solution only | | | | |
| Composition | 20% | >5.4 log | 20% | >4 log |
| according to | 40% | >5.4 log | 40% | >4 log |
| the invention | | | | |

The influence of each component of the copper solution as well as of the hydrogen peroxide solution has been gauged and validated as regards said activity on the NCTAs. Each formulation step has been subject to performance measurements according to the Western Blot method.

In a global procedure for treating objects, the performances of the composition have been assessed in vitro as regards NCTAs according to known techniques (Western Blot techniques) on at least six strains of various origins, and in vivo on animals contaminated by an infectious brain inoculum.

The composition according to the invention notably formed from a hydrogen peroxide solution mixed with an aqueous copper solution, according to The invention, thus finds an application for simultaneous disinfection and decontamination of bodies contaminated by CTAs, bacteriae, yeasts, spores, viruses and others and NCTAs such as prions. The method may advantageously exhibit a prior step of detergent stripping and/or disinfection to eliminate physically and/or enzymatically a portion of the CTA or NCTA particles.

More precisely, the procedure may thus include as a first intent, the use of a detergent and/or enzymatic or non enzymatic disinfecting detergent, for eliminating a significant portion of infectious material, quantified according to the in vitro and in vivo methods described above. In a second step, the first operation is reproduced in a more or less equivalent time and then, in a third step, the composition according to the invention is used. This procedure provides at the same time efficiency as regards NCTAs and efficiency as regards CTAs validated according to the enforced and recognised methods for measuring the performances of the disinfectants.

Naturally, other embodiments could have been contemplated by the man of the art without departing from the framework of the invention defined by the claims below.

1. The use of a composition for simultaneous disinfection and decontamination of bodies contaminated by prions and conventional transmissible agents (CTAs), such as in particular bacteriae, yeasts, viruses, mould spores, mycobacteriae, bacteria spores, said composition comprising a solution of hydrogen peroxide and an aqueous solution of copper or a derivative thereof such as copper salts, as well as at least one anionic salified surfactant compound inhibiting the decomposition of hydrogen peroxide in the presence of copper.

2. The use according to claim 1, wherein the concentration of hydrogen peroxide in the composition ranges between 5% and 9% in weight.

3. The use according to any of the claims 1 wherein said composition includes (% in weight):
anhydrous copper salts between 0.005% and 0.01%,
hydrogen peroxide between 5% and 9%,
anionic salified surfactant compound between 0.5% and 1.5%,
demineralised water q.s. 100%, the sum of the components being equal to 100%.

4. The use according to claim 1, wherein said at least one anionic salified surfactant compound is selected among the following group: dodecylbenzenesulfonate, dodecylsulfate, and sodium dodecylethersulfate.

5. The use according to claim 1, wherein said at least one anionic salified compound is ultra-pure, of synthesis quality.

6. The use according to claim 1, wherein the composition includes surfactants such as especially selected polyglucoside fatty alcohols, in particular C4 alkyls, for solubilising copper salts.

7. The use according to claim 6 wherein said composition includes polyalkoxylated fatty alcohol surfactants for assigning a hydrotopic character, in combination with a solubilising agent such as alcohols or polyols.

8. The use according to claim 1, wherein said composition comprises a colouring agent compatible with metal salts and hydrogen peroxide.

9. The use according to claim 1, wherein said composition includes (% by weight):
copper salts between 0.005% and 0.01%,
hydrogen peroxide between 5% and 9%,
anionic salified surfactant compound between 0.5% and 1.5%,
surfactants such as polyglucoside fatty alcohols between 0.1% and 0.4%.
polyalkoxylated fatty alcohol surfactant between 0.1% and 0.3%,
solubilising agent such as alcohols or polyols, between 0.1% and 0.3%,
colouring agent q.s.
water, q.s. 100%, the sum of the components being equal to 100%.

10. The use according to claim 1, wherein said composition can be prepared by extemporaneous mixture of an aqueous copper solution and of an aqueous hydrogen peroxide solution.

11. The use according to claim 10, wherein said aqueous copper solution, intended for being mixed with an aqueous hydrogen peroxide solution, further comprises said at least one anionic salified surfactant compound inhibiting the decomposition of hydrogen peroxide in the presence of copper.

12. The use according to claim 11 wherein said anionic salified surfactant compound has a content ranging between 1% and 50% (in weight) in said aqueous copper solution.

13. The use according to claim 11 wherein said aqueous copper solution includes copper salt with a content ranging between 0.1% and 0.25% (in weight).

14. The use according to claim 11 wherein said at least one anionic salified surfactant compound of the aqueous copper solution is selected among the following group: dodecylbenzenesulfonate, dodecylsulfate, and sodium dodecylethersulfate.

15. The use according to claim 11, wherein said at least one anionic salified compound of the aqueous copper solution is ultra-pure, of synthesis quality.

16. The use according to claim 11, wherein the aqueous copper solution comprises at least one surfactant such as polyglucoside fatty alcohols for solubilising copper salts, with a content ranging between 2.5 and 10% (% in weight).

17. The use according to claim 11, wherein the aqueous copper solution includes polyalkoxylated fatty alcohol surfactants for assigning a hydrotopic character, with a content ranging between 2.5% and 7.5% (% by weight).

18. The use according to claim 17 wherein the aqueous copper solution includes a solubilising agent such as alcohols or polyols with a content ranging between 4% and 7% (in weight).

19. The use according to claim 11, wherein the aqueous copper solution comprises a colouring agent compatible with metal salts and hydrogen peroxide.

20. The use according to claim 11, wherein the aqueous copper solution includes (% by weight):
copper salts between 0.1% and 0.25%
anionic salified surfactant compound between 20% and 30%,
surfactants such as polyglucoside fatty alcohols between 2.5% and 10%,
polyalkoxylated fatty alcohol surfactant 2.5% and 7.5%,
solubilising agent such as alcohols or polyols between 4% and 7%,
colouring agent q.s.,
demineralised water q.s. 100%, the sum of the components being equal to 100%.

21. The use according to claim 10, wherein said aqueous hydrogen peroxide solution to be mixed with an aqueous copper solution, for the realisation of said composition contains hydrogen peroxide stabilising agents selected among phosphonates, perborates with a content ranging between 0.002% and 0.07% in weight.

22. The use according to claim 10, wherein the aqueous copper solution is added to the aqueous hydrogen peroxide solution in 1/25 indicative proportions.

23. The use according to claim 1, for the treatment of endoscopes.

24. The use according to claim 1, wherein steps of detergent stripping and/or disinfection are performed previously for eliminating a portion, physically and/or enzymatically CTA particles or prions.
